(19) United States
(12) Patent Application Publication
(10) Pub. No.: US 2010/0093078 A1
(43) Pub. Date: Apr. 15, 2010

(54) SEPARATING DEVICE, AN ALGAE CULTURE PHOTOBIOREACTOR, AND METHODS OF USING THEM

(75) Inventors: Zhaowei Wang, Cleveland, OH (US); Joanne M. Belovich, Hinckley, OH (US)

Correspondence Address: FAY SHARPE LLP, 1228 Euclid Avenue, 5th Floor, The Halle Building, Cleveland, OH, 44115, US

(73) Assignee: Cleveland State University

(21) Appl. No.: 12/578,955

(22) Filed: Oct. 14, 2009

Related U.S. Application Data
(60) Provisional application No. 61/105,166, filed on Oct. 14, 2008.
(60) Provisional application No. 61/106,325, filed on Oct. 17, 2008.

(51) Int. Cl. C12N 5/00; C12M 1/00; C12N 1/02

(52) U.S. Cl. 435/325; 435/308.1; 435/289.1; 435/292.1; 435/261; 435/410

(57) ABSTRACT

The invention provides a device for separating a first entity and a second entity by flowing them downwardly in an inclined settling chamber. Each entity has its own outlet located at approximately the lowest end of the inclined settling chamber. The device may be used in industrial fields such as pharmaceutics, biologics, and biofuels, for the purposes of large-scale growth and separation of algae biomass, bacteria and yeast cultures; algae metabolite production; and cell separation, among others. The invention exhibits technical merits such as effective particle separation or concentration capacity, robust structure, easy operation, cost-effective manufacturability, disposability, and high productivity in e.g. perfusion photobioreactor systems.

This application claims priority to U.S. Provisional Application 61/106,325 filed on Oct. 17, 2008, and the U.S. Provisional Application 61/105,166 filed on Oct. 14, 2008, both of which are herein incorporated by reference.

### BACKGROUND OF THE INVENTION

The present invention is related to a separating or culturing device, systems including the device, and methods of using the device and the systems. In some embodiments, the invention provides a separating device such as a gravity settler, stacks of the gravity settlers, and methods of utilizing the gravity settler. These embodiments find particular application in the fields of pharmaceutics, biologics, and biofuels, for example, biological particles separation and concentration/enrichment such as large-scale cell perfusion culture, cell retention or algae culture concentration, and will be described with particular reference thereto. In other embodiments, the invention relates to a system for microorganism culture, like large scale algae culture as a photobioreactor, concentration and metabolite; and a method for utilizing such system. These embodiments find particular application in conjunction with a microorganism such as algae, and will be described with particular reference thereto. However, it is to be appreciated that the present invention is also amenable to other like applications.

Perfusion culture of suspended mammalian cells in stirred-tank bioreactors is one of the major approaches for biopharmaceutical companies to produce therapeutic and diagnostic proteins. Perfusion culture has some advantages over batch culture or fed-batch culture. In perfusion culture, high cell density and productivity can be achieved, and a constant growth environment is maintained. The wastes and product can be removed continuously from the bioreactor during the perfusion culture. The downstream processing efficiency might also be improved since the product concentration in the culture supernatant is higher than either batch or fed-batch culture.

Due to the high cell concentration, up to a 10-fold higher volumetric productivity can be achieved in a perfusion culture bioreactor compared to a fed-batch culture bioreactor. But about 90% of large-scale industrial cell culture processes are conducted in fed-batch mode. The major obstacle for the application of perfusion culture mode is the lack of effective cell retention devices, which prevents the viable cells from flowing out during perfusion culture process while spent media is removed and fresh media is added continuously. Varied approaches have been extensively discussed in, for example, Castilho, L. R.; Medronho, R. A., Cell retention devices for suspended-cell perfusion cultures. In Tools and Applications of Biochemical Engineering Science, 2002; pp 129-169; Woodside, S. M.; Bowen, B. D.; Piret, J. M., Mammalian cell retention devices for stirred perfusion bioreactors. Cytotechnology 1998, 28, 163-175; and Voisard, D.; Meuwly, F.; Ruffieux, P. A.; Baer, G.; Kadouri, A., Potential of cell retention techniques for large-scale high-density perfusion culture of suspended mammalian cells. Biotechnol. Bioeng. 2003, 82, (7), 751-765.

Moreover, the large-scale growth of algae biomass for biofuel production also requires separation of the cell biomass from the perfusion fluid. The device can be used in a photobioreactor for large scale algae culture. Large-scale growth of bacteria and yeast cultures for numerous industrial and pharmaceutical biotechnology applications also use methods for cell separation.

Due to the strict FDA regulatory requirements, it is time-consuming and costly to develop and maintain a multi-product facility based on conventional reusable bioreactors for mammalian cell culture. The clean-in-place (CIP), steam-in-place (SIP) process, and sterility validation are costly and time consuming. For cell line changes, the validation process is much longer. Single-use disposable devices have been broadly adopted by biotechnology companies in order to save time and money by avoiding extensive cleaning and validation. According to Wave Biotech Company, over 200 biotech companies use disposable Wave Bioreactors in GMP and non-GMP applications. Disposable bioreactors for cell culture have a significant advantage over conventional reusable steel and glass tank bioreactors with comparable outcome. By elimination of CIP, SIP and sterility validation process, almost three weeks turnaround time can be saved for starting a new product using disposable bioreactors. The major cost related to the disposable bioreactor after installation is the cell culture bags. A new pre-sterilized cell culture bag is used for starting a new batch culture.

The advantages of a disposable cell retention device for use with perfusion cultures using disposable bioreactors are similar to those described above. There are several disposable systems currently available commercially that promise to separate cells from perfusion fluid, with mixed success. Cell culture bags equipped with filters are commercially available for perfusion culture application. The problem associated with it is the short-lived duration, caused by filter clogging in less than two weeks from the accumulation of dead cells and cell debris. A centrifuge with a disposable insert as the cell retention device can be coupled with the disposable bioreactor. Besides potential negative impact of shear force caused by high speed rotation on cell growth and productivity, the high cost of the centrifuge system itself remains a concern.

Gravity settlers for cell retention are available commercially, but only in non-disposable systems. Vertical gravity settlers need a large volume to provide enough settling area to separate the cells from the overflow due to the slow settling velocity of animal cells. The nonworking volume of the vertical settler is too large compared to that of the bioreactor and the operating range is narrow. Inclined gravity settlers have been successfully applied to cell cultures for supporting continuous perfusion culture, as disclosed in Batt, B.; Davis, R.; Kompala, D., Inclined sedimentation for selective retention of viable hybridomas in a continuous suspension bioreactor. Biotechnol. Prog. 1990, 6, (6), 458-464; Thompson, K.; Wilson, J. Particle settler for use in cell culture. U.S. Pat. No. 5,817,505 1998; and Searles, J.; Todd, P.; Kompala, S. D., Viable cell recycle with an inclined settler in the perfusion culture of suspended recombinant chinese hamster ovary cells. Biotechnol. Prog. 1994, 10, (2), 198-206. Cell suspension is introduced into the inclined gravity settler from the lower end and overflow leaves the settler from the upper end. For example, U.S. Pat. No. 5,817,505 discloses a device for separating particles from a bulk liquid, such as viable hybridoma cells from antibody-containing liquid medium. The device comprises a plurality of settlement plates, or other surfaces, being inclined to the vertical, and a pump or other means for causing liquid containing the particles to flow upwardly over the surfaces at such a rate as to allow particles to be separated from the bulk liquid to form sediment layers on the surfaces and slide down them for collection at an appropriate point. FIG. 1 illustrates the operation of a traditional inclined gravity settler. With reference to FIG. 1, normally the angle θ is about 30°. Cell suspension from a bioreactor (not shown) is introduced into the inclined settler at position 101; underflow returns to bioreactor at position 102; and overflow leaves the settler at position 103 to a harvest tank (not shown).

The capacity of an inclined rectangular channel to retain particles can be predicted by the following equation:

S(v)=vw(L sin θ+b cos θ) (1)

where S(v) is the volumetric rate of production of fluid clarified of particles with settling velocity v, w is the width, b is the separation between the two inclined surfaces, L is the length of the settler, θ is the angle of the longitudinal axis of the gravity settler from the vertical.

Since normally L>>b, equation (1) can be simplified to:

S(v)=vwL sin θ (2)

Cell settling velocity obeys Stoke's law:

$$v = \frac{{gd}_{p}^{2} ( {ρ}_{p} - ρ )}{18 μ} \qquad ( 3 )$$

where g is the gravity constant, d is the cell diameter, ρₚ is the density of cell, ρ is the density of the culture media and μ is the viscosity of the culture medium. Equation (2) clearly shows that the larger the inclination angle, θ, the larger the cell separation capacity will be.

In traditional upward-flow inclined gravity settlers, as shown in FIG. 1, the cell suspension is fed into the bottom of the device and flows upward, while the cells settle downward, countercurrent to the flow direction. In order to facilitate dislodging of the settled cells on the lower surface, the chosen inclination angle θ was 25 to 30 degrees. According to Equation (2) much of the area of the lower cell settling surface is wasted. Besides the steep inclination, further steps were taken by cooling down the temperature of incoming cell suspension and vibrating the gravity settler body to prevent cell attachment to the lower surface of the inclined settler.

In order to increase the effective projection area, upward-flow inclined gravity settlers with multiple settling plates were developed (See U.S. Pat. No. 5,817,505; and Tabera, J.; Iznaola, M. A., Design of a lamella settler for biomass recycling in continuous ethanol fermentation process. Biotech. Bioengineering, 33, pp. 1296-1305, 1989). As shown in FIG. 5 of a review written by Voisard et al (Voisard, D.; Meuwly, F.; Ruffieux, P. A.; Baer, G.; Kadouri, A., Potential of cell retention techniques for large-scale high-density perfusion culture of suspended mammalian cells. Biotechnol. Bioeng. 2003, 82, (7), 751-765), the incoming cell suspension crosses the pathway of the settling cells from low end of the plates to the outlet port. Apparently this interference will cause some of the settling cells to reenter the multiple plate space to repeat the settling process resulting in a prolonged residence time. In order to reduce this impact, the recirculation rate is increased significantly, but this approach still increases the average cell residence time in the settler. Upward-flow multiple-plate inclined gravity settlers with stainless steel housing are manufactured by Biotechnology Solutions, Inc.

An inclined gravity settler with cell suspension feed near the center of the device, with concentrated cell stream exiting the bottom and the clarified stream exiting the top of the device has been described in Maia, A. B. R. A.; Nelson, D. L., Application of gravitational sedimentation to efficient cellular recycling in continuous alcoholic fermentation. Biotech. Bioengr., 41, 351-369, 1993.

Wang and Tan disclosed a downward-flow gravity settler with multiple inlets as shown in FIG. 2 in Chinese Patent CN00116518A (hereinafter “Wang and Tan”). FIG. 2A shows that the settler comprises multiple inlets 201, Port 202 connected to a bioreactor (not shown), and Port 203 connected to a harvest tank (not shown). FIG. 2B shows 3D illustration of the gravity settler, wherein L is the length between the selected cell inlet and outlet to bioreactor, w is the width of this rectangular gravity settler, b is the separation between the upper and lower surfaces of the settler, and θ is the angle between the longitudinal axis of the gravity settler and horizon, which is set around 55°.

The gravity settler can be operated smoothly with a 55 degree inclination angle. This downward-flow gravity settler has a capacity that is 64-94% larger (Equation 2) than that of an upward-flow cell with the same dimensions. Since the movement of settled cells is co-current with the downward flow cell suspension, the clarified supernatant facilitates the settled cells' return to the bioreactor. Pre-cooling the cell suspension and vibration of the settler body are not needed.

The downward flow inclined gravity settler in FIG. 2 is made of glass. Glass is transparent, smooth and autoclavable. The interior operation can be visually monitored. The drawback of glass is its brittleness, which makes it impractical to make a large capacity gravity settler for long-term perfusion culture systems. An accidental impact or pressure shift might break the glass wall of the settler and terminate the culture.

The raceway pond is currently the dominant method for culture of algae due to its low capital and operating costs. However, the low productivity of pond systems prevents them from being practical for large-scale algae production for biodiesel. High productivity perfusion photobioreactor systems that have low operating and construction costs are needed.

Advantageously, the present invention provides a separating device, systems comprising the device, and methods of using the device and the systems. For example, the gravity settler, the stacks thereof, and the methods of using the gravity settler as provided in the invention exhibit improved particle separation capacity, robust structure, easy operation, cost-effective manufacturability, and disposability, among other advantages. For example, the system for microorganism culture, concentration and metabolite and the method for utilizing such system as provided in the invention can satisfy the need for high productivity perfusion photobioreactor systems with low operating and construction costs.

### BRIEF DESCRIPTION OF THE INVENTION

A first aspect of the invention provides a device for separating at least a first entity and a second entity in a mixture, wherein the second entity has a higher settling speed than the first entity, comprising:

(i) an inclined settling chamber;

(ii) at least one inlet for introducing the mixture comprising the first entity and the second entity into the inclined settling chamber;

(iii) a first outlet for the first entity to exit from the settling chamber; and

(iv) a second outlet for the second entity to exit from the settling chamber;

wherein the first outlet and the second outlet are both located at approximately the lowest end of the inclined settling chamber, the first outlet and the second outlet are lower than the at least one inlet, and the second outlet is located at a position lower than the first outlet.

In various embodiments of the invention, the first entity and the second entity may be two entities with different physical, chemical and biological properties. However, the first entity and the second entity may be two entities with same physical, chemical and biological properties, such as a suspension system.

In some embodiments, the device of the invention is a gravity settler, and the first entity and the second entity in the mixture are first particles and second particles mixed in a medium.

Two or more of such gravity settlers may be combined to build a two-dimensional stack, in which the at least one inlet is located only at approximately the highest end of the stack. The term “highest end” is defined as the highest ⅓, preferably the highest ⅕, and more preferably the highest 1/10, of, for example, the stack or the inclined settling chamber.

Two or more of such two-dimensional stacks may be combined to build a three-dimensional stack, in which an upper inclined settling chamber shares a plate with a lower inclined settling chamber; and the shared plate functions as a settling surface for the upper inclined settling chamber and as an upper surface for the lower inclined settling chamber.

Another aspect of the invention provides a perfusion culture bioreactor system comprising a device selected from the gravity settler as defined above, the two-dimentional stack as defined above, or the three-dimentional stack as defined above.

Still another aspect of the invention provides a method of separating at least a first entity and a second entity in a mixture, wherein the second entity has a higher settling speed than the first entity, comprising:

(a) providing a device including an inclined settling chamber having at least one inlet, a first outlet, and a second outlet; wherein the first outlet and the second outlet are both located at approximately the lowest end of the inclined settling chamber, the first outlet and the second outlet are lower than the at least one inlet, and the second outlet is located at a position lower than the first outlet;

(b) introducing the first entity and second entity into the inclined settling chamber via the at least one inlet;

(c) flowing the first entity and second entity downwardly in the inclined settling chamber;

(d) collecting the first entity via the first outlet from the settling chamber; and

(e) collecting the second entity via the second outlet from the settling chamber.

In some embodiments, the method of the invention uses a gravity settler, and the first entity and the second entity in a mixture are first particles and second particles mixed in a medium. The second particles have a higher settling speed in the medium than the first particles.

A further aspect of the invention provides a method of separating at least first particles and second particles mixed in a medium, wherein the second particles have a higher settling speed in the medium than the first particles, comprising a step of providing and using the two-dimentional stack as defined above or the three-dimentional stack as defined above.

Another aspect of the invention provides a system for a microorganism culture and concentration comprising: (i) at least one device as defined above for microorganism culture and concentration; and (ii) at least one device as defined above for microorganism metabolite production.

In some exemplary systems, each of the devices as defined above comprises a compartment, wherein each of the compartments comprises an inclined settling chamber including:

(a) the at least one inlet for introducing the microorganism into the inclined settling chamber;

(b) the first outlet for removing an un-concentrated portion of the microorganism to exit from the settling chamber; and

(c) the second outlet for removing a concentrated portion of the microorganism from the settling chamber.

Still another aspect of the invention contemplates a method for microorganism culture, concentration and metabolite production, comprising using the system detailed above.

Still another aspect of the invention contemplates a photobioreactor with gas sparging design for microorganism, like algae, culture utilizing sunlight or artificial light as energy source for photosynthesis to produce biomass or biofuel.

### BRIEF DESCRIPTION OF THE DRAWINGS

FIG. 1 illustrates a prior art upward-flow gravity settler with an incline angle θ of about 30°;

FIG. 2A shows a prior art downward-flow gravity settler comprising multiple inlets and outlets;

FIG. 2B is a three-dimensional illustration of the gravity settler of FIG. 2A with length L between cell inlet and outlet, width w, separation between the upper and lower surfaces of the settler b, and angle between the longitudinal axis of the gravity settler and the vertical θ;

FIG. 3A schematically shows a side view of an downward-flow inclined gravity settler in an embodiment according to the invention;

FIG. 3B schematically shows a top view of the downward-flow inclined gravity settler in FIG. 3A in an embodiment according to the invention;

FIG. 4A schematically shows a side view of a two-dimensional stack of downward-flow inclined gravity settlers in an embodiment according to the invention;

FIG. 4B schematically shows a top view of the two-dimensional stack in FIG. 4A in an embodiment according to the invention;

FIG. 5 schematically shows a side view of a three-dimensional stack of multi-layer inclined gravity settler with shared plates in an embodiment according to the invention;

FIG. 6 illustrates a perfusion culture system including a stirred bioreactor, a gravity settler, and a harvest tank in an embodiment according to the invention;

FIG. 7 shows the cell retention at different entry points and perfusion rates using a gravity settler in an embodiment according to the invention;

FIG. 8 shows the average residence time of cells vs. supernatant in a gravity settler according to the invention; and

FIG. 9A shows the top view of a photo bioreactor in an embodiment of the invention;

FIG. 9B shows the side view of a photo bioreactor in an embodiment of the invention;

FIG. 10 shows a multiple gravity settler operation setup in an embodiment of the invention; and

FIG. 11 shows a photo bioreactor system setup in an embodiment of the invention.

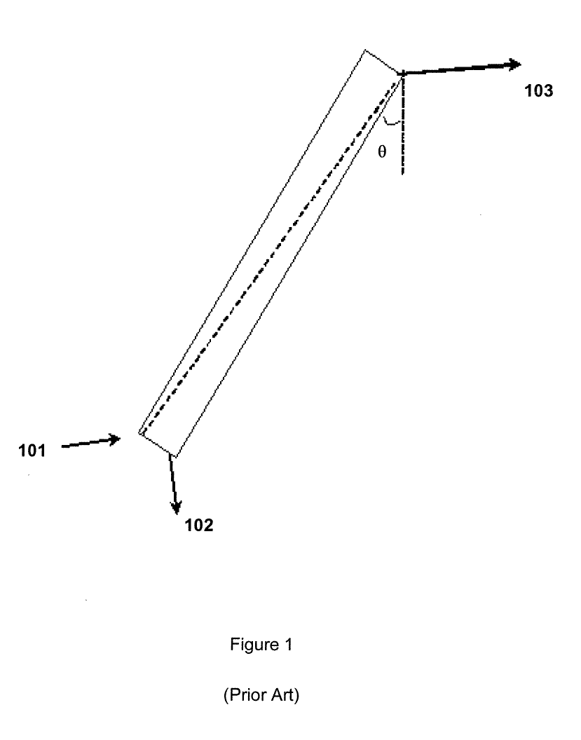

### DETAILED DESCRIPTION OF THE INVENTION

Any particular theory that is used in the description as an attempt to academically understand the mechanism of the invention, should not be interpreted as limitative to the scope of the invention.

The term “lowest end” or “lower end” is defined as the lowest ⅓, preferably the lowest ⅕, and more preferably the lowest 1/10, of, for example, the inclined settling chamber.

In a category of embodiments, the device of the invention is a gravity settler, and the first entity and the second entity in a mixture are first particles and second particles mixed in a medium. The second particles have a higher settling speed in the medium than the first particles.

When “at least a first entity and a second entity” is interpreted as “at least first particles and second particles”, the invention includes various embodiments for separating three, four, five, or more kinds of particles mixed in a medium. As second particles have a higher settling speed in the medium than first particles, similarly third particles have a higher settling speed in the medium than second particles; fourth particles have a higher settling speed in the medium than third particles; fifth particles have a higher settling speed in the medium than fourth particles; and so on and on. As a result, the gravity settler comprises:

(i) an inclined settling chamber;

(ii) at least one inlet for introducing all the particles;

(iii) a first outlet for first particles to exit from the settling chamber;

(iv) a second outlet for second particles to exit from the settling chamber;

(v) a third outlet for third particles to exit from the settling chamber;

(vi) a fourth outlet for fourth particles to exit from the settling chamber;

(vii) a fifth outlet for fifth particles to exit from the settling chamber; and on and on.

All outlets are located at approximately the lowest end of the inclined settling chamber, and are lower than the at least one inlet. In addition, fifth outlet is located at a position lower than fourth outlet, which is lower than third outlet, which is lower than second outlet, which is lower than first outlet.

The gravity settler of the invention generally has a particle separation capacity of from about 1 L/day to about 1000 L/day, preferably from about 1 L/day to about 500 L/day, and more preferably from about 1 L/day to about 200 L/day. The unit “L/day” herein is defined as the volume (liter) of the particles mixed in the medium such as a suspension, e.g. perfusion fluid, that can be separated or clarified in one day (24 hours) using the settler. In a specific embodiment, the gravity settler of the invention has a particle separation capacity of about 10 L/day.

Although there is no specific limitation to the properties of the particles such as size, the first particles and the second particles generally have a size in the range of from about 1 micron to about 50 microns, preferably in the range of from about 1 micron to about 30 microns, and more preferably in the range of from about 5 microns to about 20 microns. For example, the invention can also be used for separating small, nonbiological particles in the size range of 1-50 microns.

In exemplary embodiments, the first particles and the second particles comprise biological particles such as single-celled organisms. Examples of single-celled organisms include, but are not limited to, mammalian cells, bacteria, yeast, algae, plant cells, and any combination thereof. In some embodiments, the single-celled organisms comprise cells cultured in suspension mode such as hybridoma cells, CHO cells, and any combination thereof. However, the gravity settler of the invention may be used to separate any kind of particles, for example, non-biological particles.

In an embodiment, the particles mixed in a medium are a cell suspension of a mixture of viable and nonviable cells. Generally, the nonviable cells (as the first particles) have settling velocities that are less than that of the viable cells (as the second particles).

Any known suitable inorganic, organic, polymeric, metallic, or ceramic material may be to make the inclined settling chamber. In a preferred embodiment, the inclined settling chamber is made of light-weight, transparent, and autoclavable material, such as glass and polymeric material, for example, plastics such as polycarbonate.

In various exemplary embodiments, the inclined settling chamber has the shape of a cuboid with a length L, a width w, and a height b; and the cuboid is oriented at an angle θ from the vertical. L may be generally in the range from about 0.1 m to about 10 m, preferably in the range from about 0.1 m to about 5 m, and more preferably in the range from about 0.1 m to about 1 m. The range of w is generally from about 0.01 m to about 10 m, preferably in the range from about 0.1 m to about 5 m, and more preferably in the range from about 0.1 m to about 1 m. The range of b is generally from about 0.001 m to about 1.0 m, preferably in the range from about 0.002 m to about 0.1 m, and more preferably in the range from about 0.05 m to about 0.1 m. The range of 0 is generally from about 40 degrees to about 70 degrees, preferably in the range from about 45 degrees to about 65 degrees, and more preferably in the range from about 50 degrees to about 60 degrees.

There is no specific limitation on the number and configuration of the inlets, in preferred embodiments, the gravity settler includes a matrix of multiple inlets on the upper surface of the cuboid for introducing the at least first particles and second particles mixed in a medium into the cuboid. Optionally, the gravity settler includes an air vent in the inclined settling chamber.

When two or more of gravity settlers of the invention are combined to build a two-dimensional stack, the particle separation capacity can be improved to from about 1 L/day to about 5,000 L/day, preferably from about 1 L/day to about 1000 L/day, and more preferably from about 1 L/day to about 500 L/day, such as 200 L/day. It is contemplated that necessary structural modification and optimization may be needed for the gravity settlers to be used to build a two-dimensional stack, for example, the gravity settler can be made as a standard module that is stackable.

When two or more of two-dimensional stacks of the invention are combined to build a three-dimensional stack, the particle separation capacity can be improved to from about 1 L/day to about 10,000 L/day, preferably from about 1 L/day to about 5000 L/day, and more preferably from about 1 L/day to about 2000 L/day. It is similarly contemplated that necessary structural modification and optimization may be needed for the two-dimensional stacks to be used to build a three-dimensional stack.

In various embodiments, the invention may be used in a perfusion culture bioreactor system comprising a gravity settler, a two-dimentional stack, a three-dimentional stack, or any combination thereof. Typically, a perfusion culture bioreactor system further comprises a bioreactor such as a stirred bioreactor and a harvest tank. The products of the inventions can be manufactured for reusable use for conventional bioreactors or as disposable for disposable bioreactors for large-scale cell retention application.

In exemplary embodiments, the method of the invention is used for concentration of the first particles. For example, the first particles are particles with substantially same size which have an initial concentration C₁₁ in the medium. After the collecting of the first particles via the first outlet from the settling chamber, the first particles have a concentration C₁₂ in the medium; and C₁₂ is greater than C₁₁.

Similarly, the method of the invention may be used for concentration of the second particles. For example, the second particles may be particles with substantially the same size which have an initial concentration O₂₁ in the medium. After the collecting of the second particles via the second outlet from the settling chamber, the second particles have a concentration C₂₂ in the medium; wherein O₂₂ is greater than C₂₁.

Similarly, the method of the invention may be used for concentration of the third particles, the fourth particles, and so on.

The present invention can be broadly used in various industrial fields such as pharmaceutics, biologics, and biofuels. For example, large-scale growth of algae biomass for biodiesel production requires separation of the cell biomass from the perfusion fluid. Large-scale growth of bacteria and yeast cultures for numerous industrial and pharmaceutical biotechnology applications also use methods for cell separation. For example, the inclined gravity settler of the invention can continuously remove dead cells and cell debris from a bioreactor for long-term continuous operation.

The invention may be used the retention of viable cells during large-scale long-term perfusion cultures.

The invention affords numerous merits and benefits. For example, the gravity settlers are robust, and easy to operate. The devices are inexpensive to manufacture and thus can be made to be disposable. The efficient downward flow inclined gravity settler of the invention is estimated to need a manufacturing cost of less than $200 per 50 L/day capacity.

### Example 1

### 10 L/Day Device

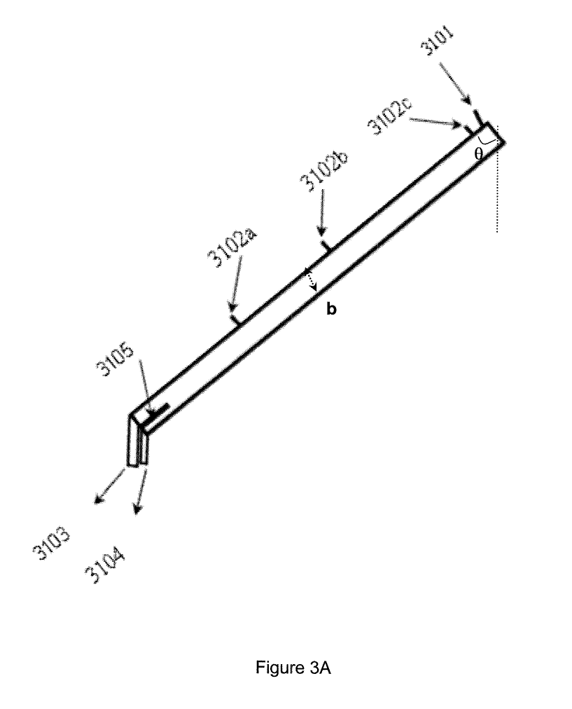

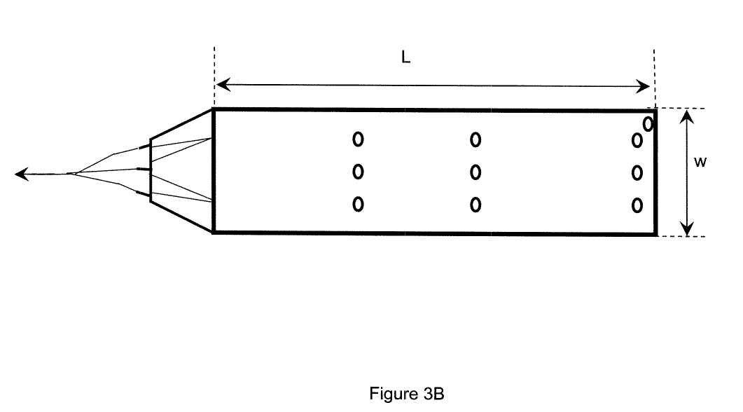

This example provided a gravity settler with a typical capacity of about 10 L/day (hereinafter “the 10 L/day device” for simplicity). FIG. 3A schematically shows the side view of the 10 L/day inclined gravity settler including air vent 3101, three inlets (3102a, 3102b, and 3102c), port 3103 to harvest, port 3104 to bioreactor, and separator 3105. Sometimes inlet 3102a, inlet 3102b, and inlet 3102c are also referred to as inlet I, inlet II, and inlet III respectively. FIG. 3B schematically shows the top view of the 10 L/day inclined gravity settler. Like Wang and Tan (which is herein incorporated by reference in its entirety), the cell separation capacity is adjusted by selecting different inlets along the longitudinal axis.

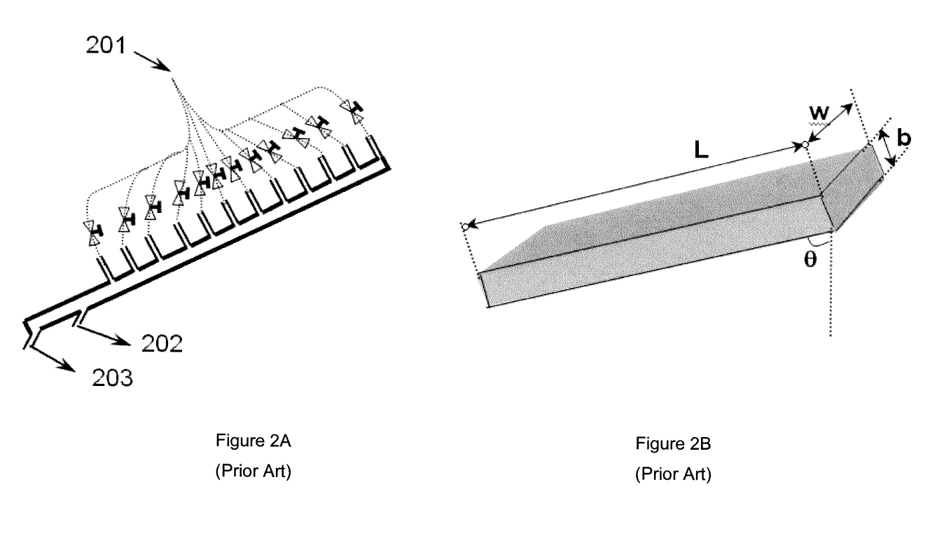

There are at least two differences between the 10 L/day device and the device in Wang and Tan. First, the outlet in Wang and Tan (as shown in FIG. 2) for cells returning to the bioreactor is located upstream of the outlet for harvest tank. In contrast, in the 10 L/day device, both outlets are located at the end of the settler and the outlet to bioreactor is underneath the outlet to harvest tank. In the device of Wang and Tan, cell accumulation sometimes occurred just above the outlet to bioreactor due to the slow flow rate at that area. There is also a concern that the slow flow rate at that point might cause the dead cells to accumulate on the surface. Another advantage of the device in this example is that that the device is stackable and more practical for supporting large scale culture.

Second, the 10 L/day device is constructed of 9.5 mm thick polycarbonate plate (McMaster, Aurora, Ohio), compared to the borosilicate glass used in Wang and Tan. Polycarbonate is light-weight compared to glass or steel. It is tough (virtually unbreakable), glass-like transparent and autoclavable. It can be extruded into desired form like many other thermoplastics. Polycarbonate sheet can be easily machined with standard metal tooling machines and is dimensionally stable. FDA compliant grade is available, which is critical for cell culture process producing therapeutical pharmaceuticals for human. It is a better material than glass for making the gravity settler provided that the cell retention efficiency is not adversely impacted. Compared to stainless steel, which is used in the commercial multiple plate settler, polycarbonate is light-weight, transparent, inexpensive and easy to manufacture.

### Example 2

### 200 L/Day Device

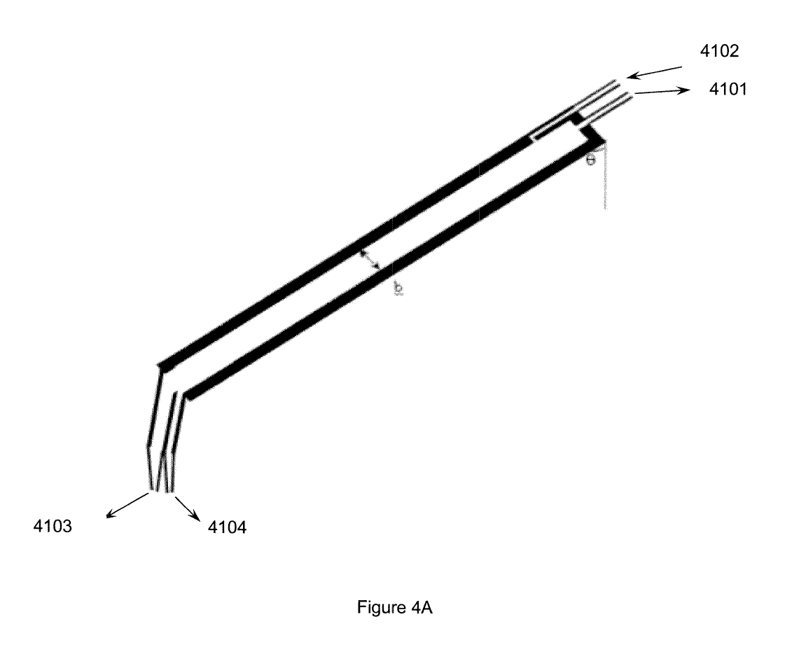

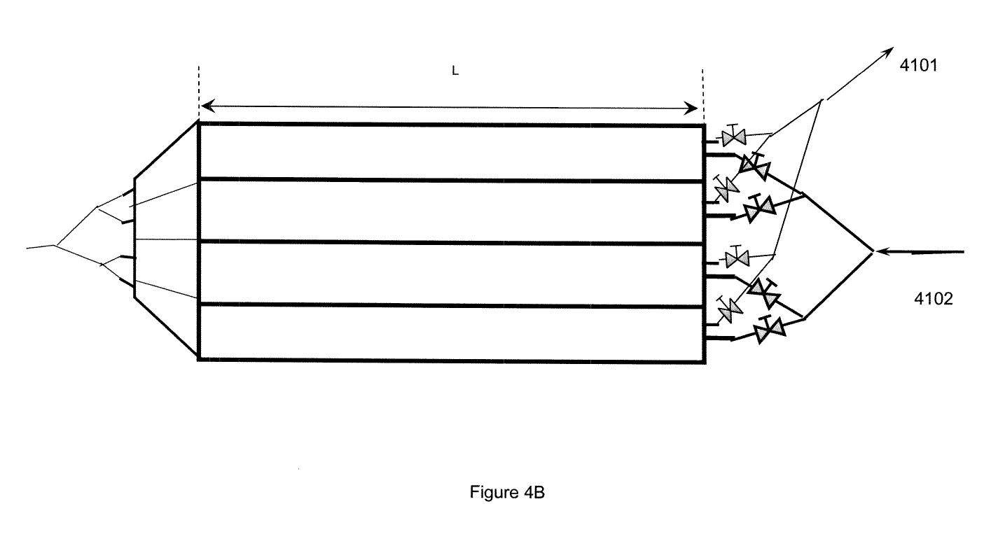

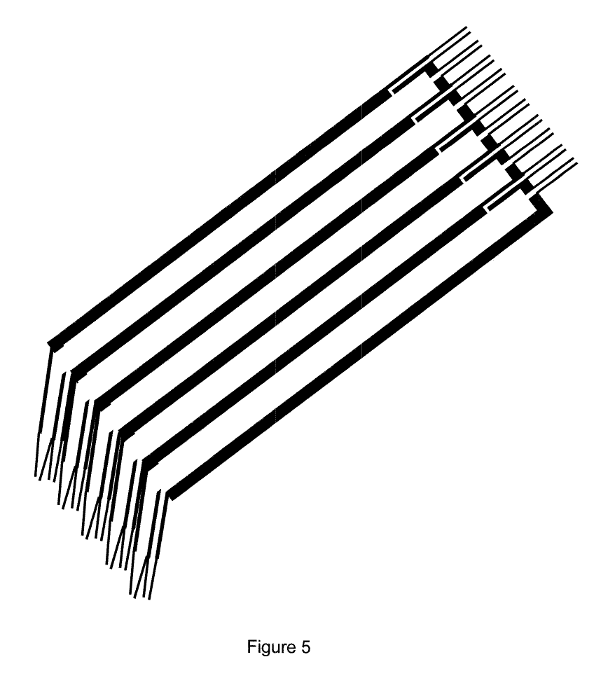

This example provided a gravity settler with a typical capacity of about 200 L/day (hereinafter “the 200 L/day device” for simplicity). This device has the same design of the outlets as the 10 L/day device. Similar to Example 1, FIG. 4A schematically shows the side view of the 200 L/day inclined gravity settler including air vent 4101, inlet(s) of cell suspension 4102, port 4103 to harvest tank, and port 4104 to bioreactor. FIG. 4B schematically shows the top view of the 200 L/day inclined gravity settler.

There are at least three differences between the 200 L/day device and the 10 L/day device. First, the 200 L/day device has inlets only at one fixed distance along the longitudinal axis near the upper end of the settler, in line with the longitudinal axis, as opposed to the multiple inlet positions along the longitudinal axis in the 10 L/day device as well as Wang and Tan. Second, the 200 L/day device has multiple channels rather than a single channel. The capacity of the settler can be modified during run-time by increasing or decreasing the number of channels in use. The example thus provides a new scale-up method comprising adding channels rather than adding length as disclosed in the Wang and Tan. Third, the 200 L/day device does not have a separator plate separating the two outlet streams. Experimental work with the 10 L/day device demonstrated that the separator is optional, for example, the separator is not necessary for the purpose of maintaining smooth flow into the nearby outlets.

### Example 3

### 1000 L/day Device

This example provided a gravity settler with a typical capacity of about 1000 L/day (hereinafter “the 1000 L/day device” for simplicity). Similar to Examples 1 and 3, FIG. 5 schematically shows the side view of the multi-layer inclined gravity settler with shared plates. A difference between this device and the 200 L/day device is that the capacity of the 200 L/day device is scaled-up to 1000 L/day by stacking several of 200 L/day settlers together. As shown in FIG. 5, the same plate is shared between two settlers, serving as the settling surface in the upper settler, and the upper surface of the lower settler. In this way, the set of settlers will occupy a smaller volume and the material cost can be reduced by almost one-half.

The three devices shown in FIGS. 3, 4, and 5 have been designed for specific working capacities and cell properties. The same design and operating principles of the inventions can be used to construct devices for a large range of working capacities and particle settling velocities.

### Example 4

### Bioreactor System and Culture Protocol

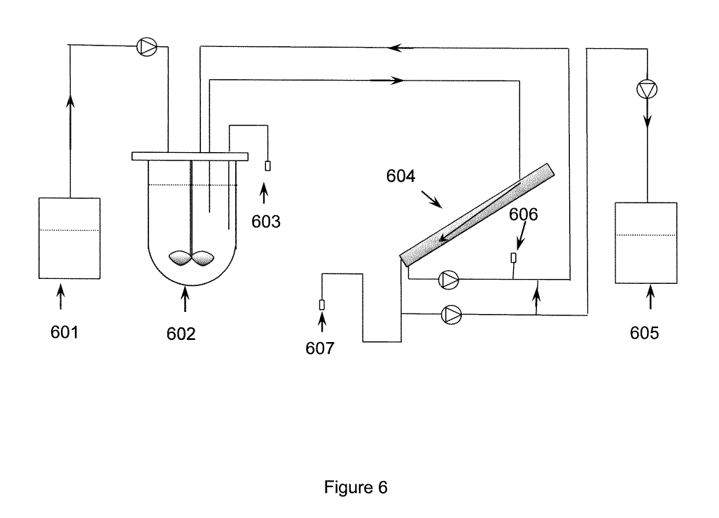

A perfusion culture system is shown in FIG. 6. Fresh medium 601 can be introduced into stirred bioreactor 602 such as a 2 L B. Braun stirred bioreactor (B. Braun biotech) with sampling port 603. Gravity settler 604 (the 10 L/Day device) is connected to stirred bioreactor 602. A sampling port 606 is used for flow from gravity settler 604 to harvest tank 605, and another sampling port 607 is used for flow from gravity settler 604 returning to stirred bioreactor 602.

HB 159 cells in exponential growth phase were inoculated in the stirred bioreactor. The culture was started as batch culture followed by perfusion in order to achieve high cell concentration for the short-term recycle cell retention test. The real perfusion amount was 1 L/day for the perfusion bioreactor. Viable cell density was maintained over 1×10⁷ cells/mL in the bioreactor. The flow rate from the outlet port connecting to harvest tank 605 is taken as virtual perfusion amount. It is called virtual perfusion amount because the system is not really perused with that amount of fresh media but it can show the real capacity that the retention device can process. For simplicity, the term “perfusion amount” is used in place of “virtual perfusion amount”.

The cell retention rate, R, is defined as:

$$R = \frac{{X}_{R} - {X}_{O}}{{X}_{R}} \times 100 \% \qquad ( 4 )$$

where X_{R} is the cell concentration in the bioreactor; X_{O} is the cell concentration in the overflow stream that exits the gravity settler via the port to the harvest tank.

The residence time of clarified supernatant in the gravity settler is determined using Equation (5), where τᵢ is residence time; Q is volumetric flow rate of cell suspension through the gravity settler and V is the working volume of the gravity settler:

$${τ}_{1} = \frac{V}{Q} \qquad ( 5 )$$

The cell residence time residence time, τ₂, was measured when the separation process reached steady status. The flows into the gravity settler and out via the port to harvest tank were temporarily shut off and the cell suspension was completely collected via the port to bioreactor after vigorous shaking of the gravity settler. The cell concentration in the gravity settler and in bioreactor was measured respectively. The cell residence time was calculated using Equation (6), where X_{G} is cell concentration in the gravity settler.

$${τ}_{2} = {τ}_{1} \frac{{X}_{G}}{{X}_{R}} \qquad ( 6 )$$

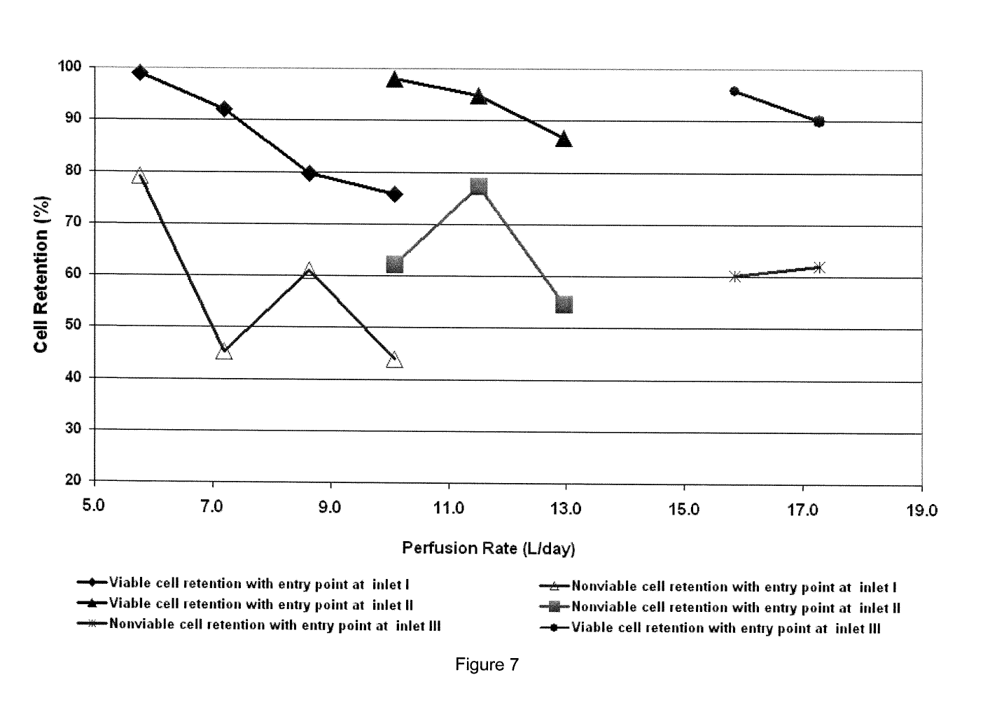

FIG. 7 shows the cell retention at different entry points and perfusion rates. Cell retention capability was not strictly proportional to the length L, as predicted by Equation (2). As shown in Table 1, the length for Inlet 3102c is 3.5 times of that for the Inlet 3102a. When the perfusion amount for Inlet 3102c is 15.8 L/day, 2.7 times of that for the Inlet 3102a, the viable cell retention rate was 3% less than that of the Inlet 3102a.

TABLE 1
Viable cell retention rate at different distances between the entry
point and lower end of the separator, L, and perfusion rates

| | Inlet 3102a | Inlet 3102b | Inlet 3102c |
|---|---|---|---|
| L (cm) | 16 | 33.5 | 57 |
| Perfusion amount (L/day) | 5.8 | 10.8 | 15.8 |
| Viable cell retention rate (%) | 99 | 98 | 96 |

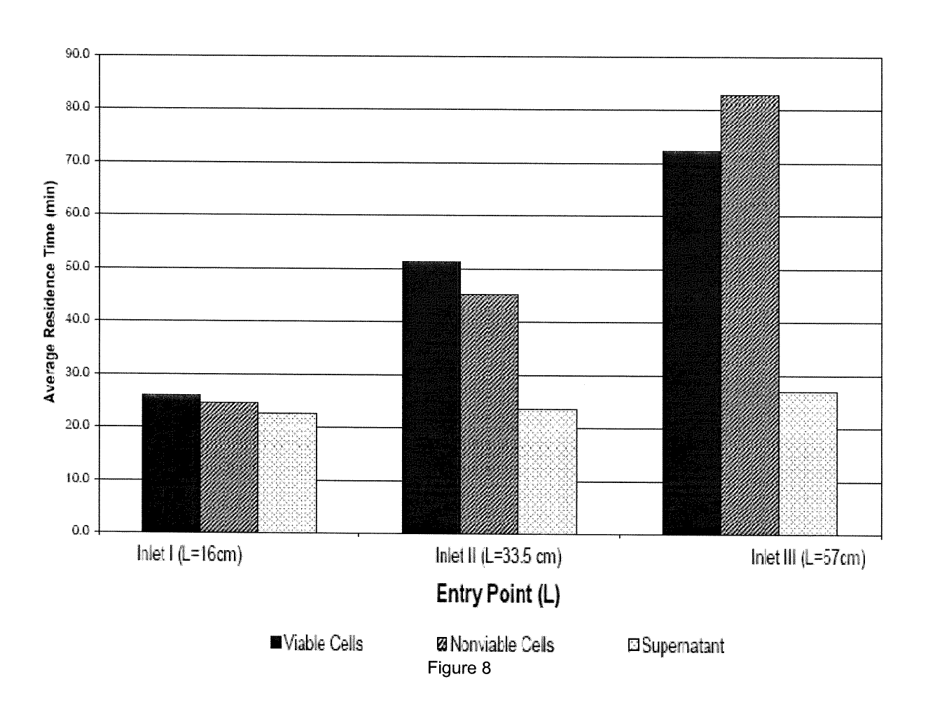

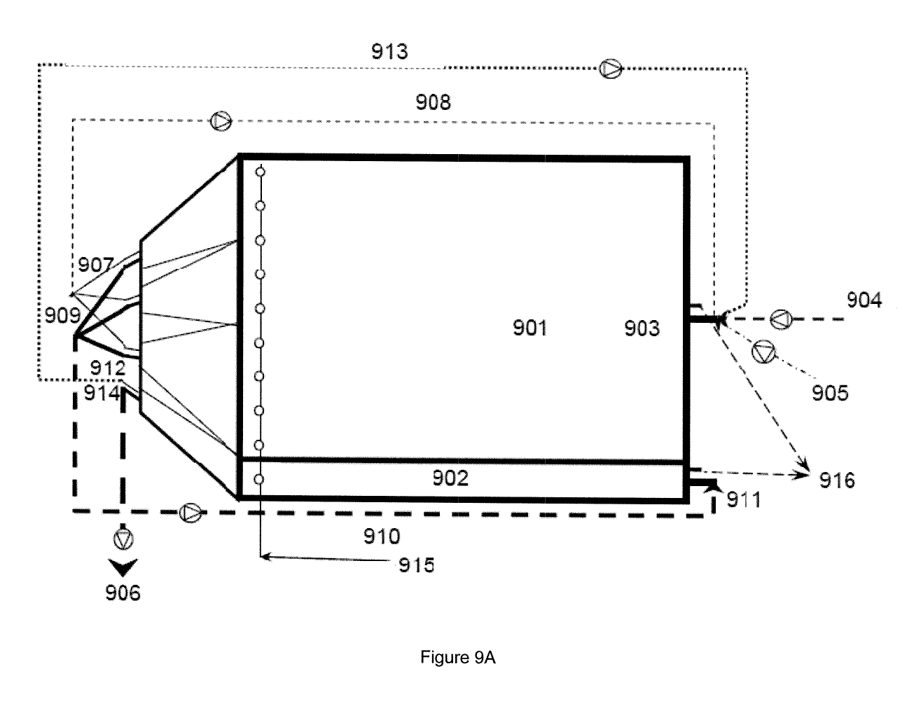

It is believed that the discrepancy was caused by the different cell residence times in the gravity settler as shown in FIG. 8, which shows the average residence time of cells vs. supernatant. When the supernatant residence was almost unchanged, the cell residence time was approximately proportional to the length. This suggests that the sliding speed of the settled cells stays constant while the cell suspension or supernatant increases along with the perfusion amount. The longer residence time of settled cells will cause cell accumulation in the settler. Upper layers of settled cells will be dragged by the hydraulic force due to the difference of speed between settled cells and the fluid. From direct visual observation through the transparent upper surface of the gravity settler, the settled cells slide down like traveling dune. The uneven distribution would induce turbulence in the laminar flow, and then reduce the cell retention efficiency. This result indicates that inclined gravity settler cannot be linearly scaled up by simply increasing the length, which would lower the cell retention efficiency per unit area. The ideal design should make the settled cells move at the similar speed as the supernatant, as in the situation where the perfusion amount was 5.8 L/day with Inlet I as entry point.

Theoretically the downward movement speed of the settled cells should be constant for a given inclination angle. Therefore to maintain the cell retention efficiency, the depth, d, of the settler should be increased in proportion to the increase in the working length of the settler. The maximum depth of the gravity settler is limited to prevent the working volume of the gravity settler from being too large compared to the bioreactor working volume. However the width, w, can be increased independently of the depth. Therefore, increasing the width is important for efficient scale-up.

This test result implies that the design in Wang and Tan is not suitable to scale up by simply increasing the working length without changing the settler depth for large scale culture. Due to limitations on the practical depth, the present invention provides a new design principle involving a change in the working width of the settler.

The results of the culture tests show that the 10 L/day gravity settler is a reliable cell retention device for large scale high-density perfusion culture applications with improved performance and flexibility. To increase the capacity for very large-scale systems (200-1000 L/day), a multiple channel and/or multiple plate design was adopted, which still allows flexibility in capacity requirements during real-time operation.

In another category of embodiments, the system of the invention for a microorganism culture and concentration comprises (i) at least one device as defined above such as a compartment, which is used for microorganism culture and concentration; and (ii) at least one device as defined above such as a compartment, which is used for microorganism metabolite production. The microorganism may comprise algae, and the metabolite may comprise an oil-precursor such as lipid.

In some exemplary systems, each of the two compartments comprises an inclined settling chamber which is comprised of:

(a) at least one inlet for introducing the microorganism into the inclined settling chamber;

(b) a first outlet for removing a first entity such as an un-concentrated portion of the microorganism to exit from the settling chamber; and

(c) a second outlet for removing a second entity such as a concentrated portion of the microorganism from the settling chamber.

An “un-concentrated portion of the microorganism” is a portion of the microorganism that has a concentration lower than the overall or average concentration of the microorganism in the entire compartment or chamber, which includes the concentration of zero (e.g. the clear medium in a suspension). A “concentrated portion of the microorganism” is a portion of the microorganism that has a concentration higher than the overall or average concentration of the microorganism in the entire compartment or chamber.

In various exemplary embodiments, the system further comprises a light source such as the sun, and the compartment for microorganism culture and concentration is a photo-bioreactor.

In various exemplary embodiments, the first outlet for un-concentrated portion of the microorganism to exit from the settling chambers of the compartment for microorganism culture and concentration is connected to the at least one inlet for introducing the microorganism into the inclined settling chamber of the compartment for microorganism culture and concentration.

In various exemplary embodiments, the second outlet for concentrated portion of the microorganism to exit from the settling chamber of the compartment for microorganism culture and concentration is connected to the at least one inlet for introducing the microorganism into the inclined settling chamber of the compartment for microorganism metabolite production.

In various exemplary embodiments, the first outlet for un-concentrated portion of the microorganism to exit from the settling chambers of the compartment for microorganism metabolite production is connected to the at least one inlet for introducing the microorganism into the inclined settling chamber of the compartment for microorganism culture and concentration.

In various exemplary embodiments, the second outlet for concentrated portion of the microorganism to exit from the settling chamber of the compartment for microorganism metabolite production is connected to a harvest tank for the extraction of the metabolite.

In various exemplary embodiments, the system of the invention includes multiple (for example 9) compartments for microorganism culture and concentration and one compartment for microorganism metabolite production.

In various exemplary embodiments, each of the inclined settling chambers has the shape of a cuboid with a length L, a width w, and a height h; and the cuboid is oriented at an angle θ from the vertical; wherein L is in the range from about 0.1 m to about 10 m, w is in the range from about 0.01 m to about 10 m, h is in the range from about 0.001 m to about 1.0 m, and θ is in the range from about 10 degrees to about 80 degrees.

In various exemplary embodiments, each of the inclined settling chambers has a gas vent. The compartment for microorganism culture and concentration may be a perfusion culture bioreactor. The compartment for microorganism culture and concentration may be a gas sparged bioreactor. Each of the inclined settling chambers may have gas delivery ports at the lower end. For example, a settling chamber may include a gas sparging inlet on the top surface of the lower end of the settler for algae culture application.

In specific exemplary embodiments, the invention provides a two stage photobioreactor for algae culture using at least one compartment for microorganism culture and concentration and at least one compartment for microorganism metabolite production. Each stage has the general design of an inclined gravity settler. The invention separates the growth phase and oil accumulation phase in two different places. Algae are cultured in the first stage in an environment for fast growth, where nitrogen supply is sufficient. Following this stage, the concentrated algae culture is introduced into the second stage, in which nitrogen is depleted to promote oil accumulation. The design of the second chamber also serves to partially dewater the culture.

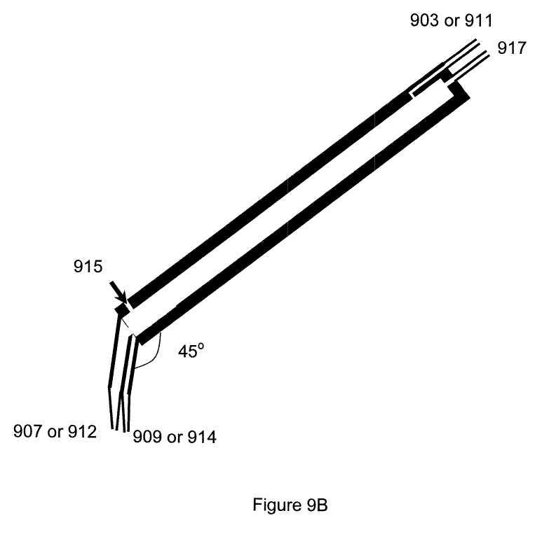

As shown in FIG. 9A, the system comprises a compartment for microorganism culture and concentration such as a growth chamber 901, and a compartment for microorganism metabolite production such as oil generation chamber 902. The first outlet(s) 907 for un-concentrated portion of the microorganism to exit from the chambers 901 is connected via channel 908 to the inlet 903 for introducing the reduced concentration of algae culture back into chamber 901 for further culture and concentration. The second outlet(s) 909 for concentrated portion of the microorganism to exit from the chamber 901 is connected via channel 910 to the inlet 911 for introducing the microorganism into the chamber 902 for microorganism metabolite production. The first outlet 912 for un-concentrated portion of the microorganism to exit from the chamber 902 is connected via channel 913 to the inlet 903 for introducing the microorganism into the chamber 901 for further culture and concentration. The second outlet 914 for concentrated portion of the microorganism to exit from the chamber 902 is connected via channel 906 to a harvest tank (not sown) for the extraction of the metabolite such as lipid. Each chamber may have a gas vent such as gas inlets 915 and gas outlets 916. Fresh media 905 can be fed into the chamber 901 via the inlet 903. The growth medium fed into the growth chamber 901 is preferably designed such that it has sufficient nitrogen for the microorganism growth, but it is depleted upon exiting from the growth chamber 901. Only additional carbon dioxide is fed as a nutrient into the chamber 902.

To concentrate the algae culture about nine-fold in the growth chamber 901, the design can be that the ratio of the width of the growth chamber 901 to the width of the oil accumulation chamber 902 to be 9:1 (providing all other dimension parameters are the same). The algae concentration exiting the oil generation (accumulation) chamber 902 to the harvest tank (not shown) is expected to be 81-fold that of the concentration of the initial algae inoculum 904 entering the inlet 903 of the chamber 901, without even considering the increase due to cell growth in the chamber 902. This concentration factor is in the same range of that expected with use of centrifuge.

If cell growth in the chamber 901 is taken into consideration, the concentration of the outflow in channel 906 from the chamber 902 to a harvest tank (not shown) will be over 100-fold (assuming the algae doubling time is about 24 hours) compared to the initial algae inoculum 904 entering the inlet 903 of the chamber 901.

With reference to FIG. 9B, which is a part of the cross-sectional view of FIG. 9A, the system includes the microorganism inlet 903 or 911, the first outlet 907 or 912, the second outlet 909 or 914, the gas inlet 915, and the gas outlet 917.

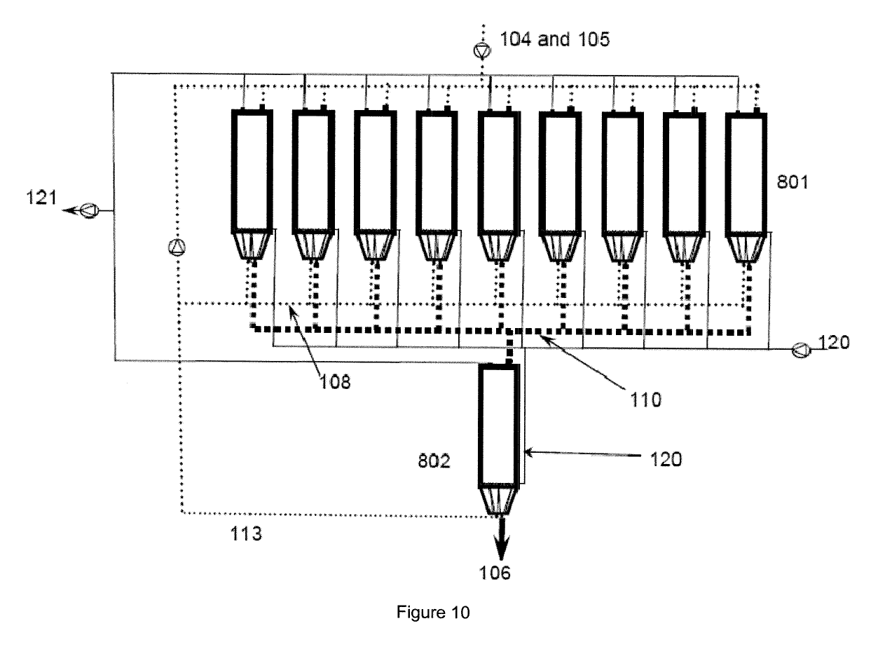

FIG. 10 illustrates a system similar to FIG. 9A. With reference to FIG. 10, the system includes 9 growth chambers 801 and one oil generation chamber 802. The initial inoculum 104 and fresh media 105 can be fed into each of chambers 801 through channels represented as circularly-dotted lines above the chambers 801. Reduced concentration of algae culture exiting from chambers 801 can also be fed into each of chambers 801 through channel(s) 108 under the chambers 801. Concentrated one time algae culture exiting from chambers 801 can be fed into the chamber 802 through the channel 110. Reduced concentration of algae culture from chamber 802 can be transferred to chambers 801 through channel 113. Final concentrated stream from chamber 802 was delivered to a harvest tank (not shown) for oil extraction via channel 106. Gas can enter all chambers 801 and 802 through channels 120 and exit from chambers 801 and 802 through channels 121.

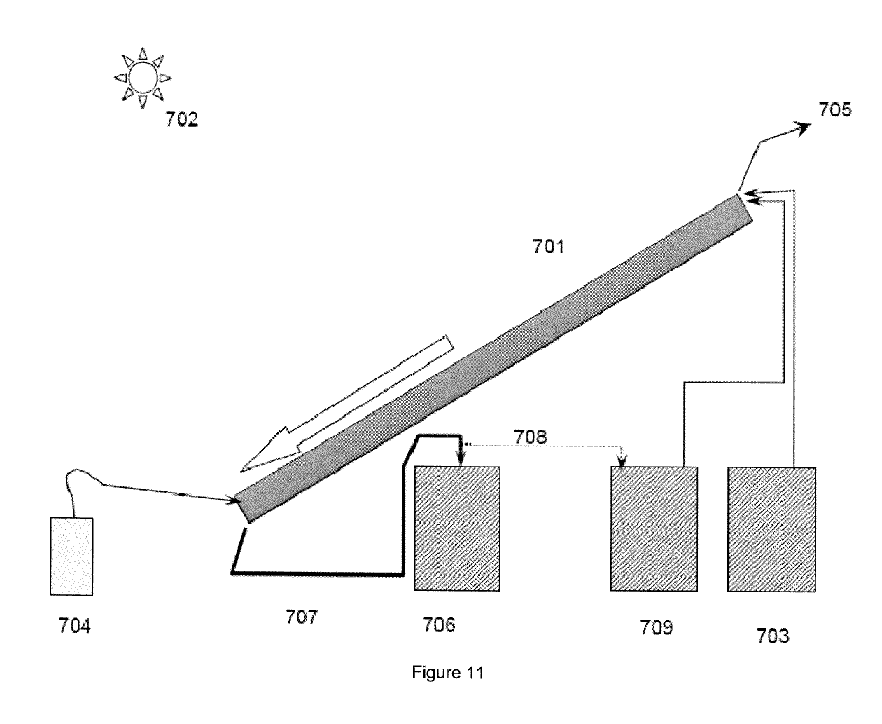

FIG. 11 illustrates an entire operational system applying the two-stage algae photobioreactor for oil production. The details of a photobioreactor 701 can be similar to FIGS. 9A and 10, which is under the illumination of a light source 702 such as the sun. Fresh media tank 703 provides needed media to bioreactor 701, and gas tank 704 deliveries gas to bioreactor 701, which can exit from bioreactor at outlet 705. Final concentrated stream can be transferred to a harvest tank 706 for oil via channel 707. Algae culture to be collected for night storage can be transferred from tank 706 to a night storage tank 709 via channel 708, which can be later (e.g. during daytime) fed into photo-bioreactor 701 for further utilization.

The invention exhibits numerous advantages compared to pond culture procedure. For example, unlike the even distribution of nutrients in the raceway pond culture, the nitrite-sufficient and the nitrite-deprived environments are separated in the photobioreactor. The stream leaving the 2nd stage to the harvest tank, has been highly concentrated due to the effect of the gravity settler. Conversely with raceway ponds, the harvest stream must then be concentrated with a flocculation process and centrifuge, which introduce additional costs. In the photobioreactor system, the carbon dioxide can be used more efficiently compared to pond culture since the gas is fed into a closed environment, resulting in less loss to the environment. For same reason, oxygen inhibition can be reduced in the photobioreactor compared to ponds by continuous pumping out in the photo bioreactors. Greater efficiency of sunlight use is obtained in the photobioreactors compared to ponds due to the inclination angle of the photobioreactor, which maximizes sunlight collection. Less land is needed due to improved productivity using photobioreactors. A closed photobioreactor has lower chance of contamination from native, low-oil producing algae strains. Water use is minimized in a photobioreactor system compared to a pond where significant evaporation takes place.

The exemplary embodiments have been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A device for separating at least a first entity and a second entity in a mixture, wherein said second entity has a higher settling speed than said first entity, comprising:
(i) an inclined settling chamber;
(ii) at least one inlet for introducing said mixture into said inclined settling chamber;
(iii) a first outlet for said first entity to exit from the settling chamber; and
(iv) a second outlet for said second entity to exit from the settling chamber;
wherein said first outlet and said second outlet are both located at approximately the lowest end of said inclined settling chamber, said first outlet and said second outlet are lower than said at least one inlet, and said second outlet is located at a position lower than said first outlet.

2. The device according to claim 1, which is a gravity settler, wherein the first entity and the second entity in a mixture are first particles and second particles mixed in a medium.

3. The device according to claim 2, which has a particle separation capacity of from 1 L/day to 1000 L/day.

4. The device according to claim 1, wherein the settling chamber includes a gas sparging inlet on the top surface.

5. The device according to claim 2, in which the first particles and the second particles have a size in the range of from about 1 micron to about 50 microns.

6. The device according to claim 2, in which the first particles and the second particles comprise non-biological particles.

7. The device according to claim 2, in which the first particles and the second particles comprise biological particles.

8. The device according to claim 7, in which the biological particles comprise single-celled organisms.

9. The device according to claim 8, in which the single-celled organisms are selected from mammalian cells, bacteria, yeast, algae, plant cells, and any combination thereof.

10. The device according to claim 8, in which the single-celled organisms comprise cells cultured in suspension mode such as hybridoma cells, CHO cells, and any combination thereof.

11. The device according to claim 2, in which the settling chamber is comprised of glass or a polymeric material.

12. The device according to claim 2, in which the inclined settling chamber has the shape of a cuboid with a length L, a width w, and a height h; and the cuboid is oriented at an angle θ from the vertical; wherein L is in the range from about 0.1 m to about 10 m, w is in the range from about 0.01 m to about 10 m, h is in the range from about 0.001 m to about 1.0 m, and θ is in the range from about 40 degrees to about 70 degrees.

13. The device according to claim 12, which includes a matrix of multiple inlets on the upper surface of the cuboid for introducing said at least first particles and second particles mixed in a medium into the cuboid.

14. The device according to claim 2, further including an air vent in the inclined settling chamber.

15. A two-dimensional stack comprising two or more gravity settlers according to claim 2, in which the at least one inlet is located only at approximately the highest end of the settling chamber.

16. A three-dimensional stack comprising two or more stacks according to claim 16, in which an upper inclined settling chamber shares a plate with a lower inclined settling chamber; and the shared plate functions as a settling surface for the upper inclined settling chamber shares and as an upper surface for lower inclined settling chamber.

17. A perfusion culture bioreactor system comprising a device selected from the gravity settler according to claim 2.

18. The perfusion culture bioreactor system according to claim 17, further comprising a stirred bioreactor and a harvest tank.

19. A method of separating at least a first entity and a second entity in a mixture, wherein said second entity has a higher settling speed than said first entity, comprising:
(a) providing a device which comprises (i) an inclined settling chamber; (ii) at least one inlet; (iii) a first outlet; and (iv) a second outlet; wherein said first outlet and said second outlet are both located at approximately the lowest end of said inclined settling chamber, said first outlet and said second outlet are lower than said at least one inlet, and said second outlet is located at a position lower than said first outlet;
(b) introducing said first entity and second entity into said inclined settling chamber via said at least one inlet;
(c) flowing said first entity and second entity downwardly in said inclined settling chamber;
(d) collecting said first entity via said first outlet from the settling chamber; and
(e) collecting said second entity via said second outlet from the settling chamber.

20. The method according to claim 19, wherein the first entity and the second entity in a mixture are first particles and second particles, and the method is used for concentration of the first particles; wherein the first particles are particles with substantially same size which have an initial concentration C₁₁ in the medium; after the collecting of said first particles via said first outlet from the settling chamber the first particles have a concentration C₁₂ in the medium; and C₁₂ is greater than C₁₁.

21. The method according to claim 19, wherein the first entity and the second entity in a mixture are first particles and second particles, and the method is used for concentration of the second particles; wherein the second particles are particles with substantially same size which have an initial concentration C₂₁ in the medium; after the collecting of said second particles via said second outlet from the settling chamber the second particles have a concentration C₂₂ in the medium; and C₂₂ is greater than C₂₁.

22. A system for a microorganism culture and concentration comprising: (i) at least one device according to claim 1 for microorganism culture and concentration; and (ii) at least one device according to claim 1 for microorganism metabolite production.

23. The system according to claim 22, wherein the metabolite comprises an oil-precursor.

24. The system according to claim 23, wherein the oil-precursor comprises lipid.

25. The system according to claim 22, further comprising a light source, wherein the device for microorganism culture and concentration is a photo-bioreactor.
